(19) United States
(12) Patent Application Publication
(10) Pub. No.: US 2024/0365760 A1
(43) Pub. Date: Nov. 7, 2024

(54) TRANSGENIC ANIMAL MODEL, METHOD FOR CONSTRUCTING SAME, AND USE THEREOF

(71) Applicant: SHENZHEN INSTITUTES OF ADVANCED TECHNOLOGY CHINESE ACADEMY OF SCIENCES, SHENZHEN (CN)

(72) Inventors: DI CHEN, SHENZHEN (CN); DAN YI, SHENZHEN (CN)

(21) Appl. No.: 18/650,088

(22) Filed: Apr. 30, 2024

Related U.S. Application Data
(63) Continuation of application No. PCT/CN2022/097333, filed on Jun. 7, 2022.

(51) Int. Cl. A01K 67/0276; C12Q 1/686; C12Q 1/6883

(52) U.S. Cl. CPC A01K 67/0276; C12Q 1/686; C12Q 1/6883; A01K 2217/15; A01K 2227/105; A01K 2267/03

(57) ABSTRACT

Provided are a transgenic animal model, a method for constructing same, and use thereof. An Axin1flox/flox transgenic mouse is used to mate with an Agc1-CreER transgenic mouse to produce an Axin1+Agc1ER condition knockout mouse. Then by means of tamoxifen induction, the Axin1 expression in the Axin1+Agc1ER condition knockout mouse is down-regulated, and a disease combined model with the pathological characteristics of “growth plate chondrocyte hypertrophy, heterotopic ossification and articular cartilage degeneration of knee joints” is successfully established. The model is similar to clinically related pathological features, and can be used for systematically studying the pathogenesis of growth plate chondrocyte hypertrophy, heterotopic ossification and osteoarthritis and further screening for drugs for treating related diseases.

### CROSS REFERENCE TO RELATED APPLICATIONS

The present disclosure is a U.S. continuation of co-pending International Patent Application Number PCT/CN2022/097333, filed on Jun. 7, 2022. The entire contents of the above-identified applications are incorporated herein by reference.

### SEQUENCE LISTING

The instant application contains a Sequence Listing which has been submitted electronically in ASCII format and is hereby incorporated by reference in its entirety. Said ASCII copy, created on Jul. 9, 2024, is named US18650088 SEQ.xml and is 4,889 bytes in size.

### TECHNICAL FIELD

The present invention belongs to the field of biotechnology and specifically relates to a transgenic animal model, a method for constructing same, and use thereof, in particular to a transgenic animal model for growth plate chondrocyte hypertrophy, ectopic ossification, and osteoarthritis, a method for constructing same, and use thereof.

### BACKGROUND

Skeletal development of a mammal begins with formation of transparent cartilages during a embryonic stage. Osteoblasts from a periosteum are distributed around the transparent cartilages, forming dense bones and forming diaphyses and spongy bones, known as primary ossification centers. Osteoclasts from a hematopoietic system destroy the spongy bones and form medullary cavities. With continuous formation of the medullary cavities (calcification), after birth of the mammal, secondary ossification centers are formed at two poles of axis posterior primitive transparent cartilage (epiphyses). After the secondary ossification centers are formed, cartilages will be replaced by sponge bones, but intact articular cartilages and growth plates (epiphyseal plates) composed of chondrocytes will be retained. Continuous proliferation and hypertrophy of chondrocytes lead to postnatal bone calcification and development. Unlike humans, proliferation and calcification of chondrocytes in growth plates of mice do not terminate. Therefore, we have the opportunity to investigate the role of growth plates in bone development and postnatal bone growth of mice.

Heterotopic ossification (HO) is a process of bone tissue formation in exoskeleton muscles and soft tissues, commonly seen in patients with neurological paralysis. In clinical practice, HO is more commonly seen in traumatic ectopic ossification (traumatic ossifying myositis), progressive ossifying fibrodysplasia (FOP), and progressive heterotopic bone hyperplasia (POH). Patients' muscles appear warm, soft and firm swelling, and motion ranges of joints served by relevant muscles decrease. The incidence rate varies according to differs in reasons of the disease, and specific molecular pathological mechanism is not clear. At present, there is no clear treatment method other than anti-inflammatory drugs, surgical resection, and radiation therapy.

Osteoarthritis (OA) has affected over 100 million patients in our country, seriously affecting life quality of the elderly. OA is a bone joint degenerative disease caused by interaction of systemic susceptibility factors and local mechanical factors, characterized by articular cartilage degeneration. Main clinical manifestations of OA are pain, swelling, deformity, and limited mobility of affected joints, and imaging features include narrowing of joint gaps and formation of osteophytes around subchondral bones. At present, the treatment for OA is mainly limited to passive, conservative, or even traumatic surgical treatments to alleviate symptoms and correct deformities. This is mainly due to limited understanding of molecular mechanisms of OA, resulting in a lack of effective anti-OA treatments targeting the etiology.

Due to ethical limitations, medical research cannot perform research on patients with knee osteoarthritis, therefore animal research occupies an important position.

### SUMMARY OF THE DISCLOSURE

The purpose of the present invention is to overcome the problem of the inability to establish a model that combines multiple diseases in existing technology.

In view of this, the present invention provides a method for constructing a transgenic animal model, comprising the following steps: S1, constructing Axin1^{flox/flox} transgenic mice and Agc1-CreER transgenic mice; S2, selecting adult Axin1^{flox/flox} mice and Agc1-CreER mice to mate, and producing Axin1^{Agc1ER} condition knockout mice by mating; S3, injecting tamoxifen into abdominal cavities of adult Axin1^{Agc1ER} condition knockout mice, with the injection continuing for 5 days; S4, regularly detecting biochemical indicators of the Axin1^{Agc1ER} condition knockout mice during modeling, and performing histological and histomorphometric analyses; S5, when a mouse's body simultaneously experiences growth plate chondrocyte hypertrophy, ectopic ossification, and knee joint cartilage degeneration syndrome, obtaining the transgenic animal model.

Specifically, the step S2 specifically comprises: mating adult female Axin1^{flox/flox} transgenic mice with adult male Agc1-CreER transgenic mice, or mating adult male Axin1^{flox/flox} transgenic mice with adult female Agc1-CreER transgenic mice; when newborn mice grow to 2-3 weeks, taking 1-3 mm tail tips of the mice for mice genotype identification; adding lysis solution into tissue needing lysis, and incubating in a water bath after vortex oscillation; after thoroughly mixing evenly by vortex oscillation, taking supernatan to obtain lysates product; performing PCR amplification for Axin1 and CreER in the lysates; by agarose gel electrophoresis, detecting newborn mice corresponding to the lysates of Axin1 and CreER amplification bands as the Axin1^{Agc1ER} condition knockout mice.

Specifically, the above lysis solution comprises 2 μl Tris-Hcl with a pH of 7.5, 20 μl 0.5M EDTA with a pH of 8.0, 6 μl 5M NaCl, 20 μl SDS with a mass to volume ratio of 10%, and 1 μl proteinase K with a concentration of 10 mg/ml.

Specifically, primer used in the above PCR amplification include Axin1 forward primer, Axin1 reverse primer, CreER forward primer, and CreER reverse primer; a sequence of the Axin1 forward primer is set forth in SEQ ID No. 1; a sequence of the Axin1 reverse primer is set forth in SEQ ID No. 2; a sequence of the CreER forward primer is set forth in SEQ ID No. 3; a sequence of the CreER reverse primer is set forth in SEQ ID No. 4.

Specifically, in the step S3, the injection amount of tamoxifen is calculated based on the body weight of the Axin1^{Agc1ER} condition knockout mice, injecting 1 mg per 10 g.

Specifically, in the step S4, the biochemical indicators comprise expression levels of Ainx1, Aixn2, β-catenin, COl-X, MMP13, and Ki-67 in the bodies of the mice.

Specifically, in the step S4, the histomorphometric analyses comprise expression of micro-CT and TRAP staining of the mice.

In addition to the above detection indicators and methods, technical personnel in this field may choose other detection indicators and methods according to actual needs, all of which fall within the scope of protection of the present application.

The transgenic animal model constructed by the constructing method provided by the present invention downregulates the expression of Axin1 gene, and is complicated by transgenic animal models with growth plate chondrocyte hypertrophy, ectopic ossification, and osteoarthritis. It can be used to screen drugs for the treatment of growth plate chondrocyte hypertrophy, ectopic ossification, and osteoarthritis diseases. It can also be used for systematic research on the pathogenesis of growth plate chondrocyte hypertrophy, ectopic ossification, and osteoarthritis.

Compared with the prior art, the present invention has the following advantages and beneficial effects.

The method for constructing the transgenic animal model provided by the present invention uses Axin1^{flox/flox} transgenic mice to mate with Agc1-CreER transgenic mice to produce Axin1^{Agc1ER} condition knockout mice. Then by means of tamoxifen induction, the Axin1 expression in the Axin1^{Agc1ER} condition knockout mice is down-regulated, and a disease combined model with the pathological characteristics of “growth plate chondrocyte hypertrophy, heterotopic ossification and articular cartilage degeneration of knee joints” is successfully established. The growth plate chondrocyte hypertrophy, ectopic ossification, and osteoarthritis features of the animal model of the Axin1^{Agc1ER} condition knockout mice constructed using the method provided by the present invention is similar to clinically related pathological features, and can be used for systematically studying the pathogenesis of growth plate chondrocyte hypertrophy, heterotopic ossification, and osteoarthritis, and further screen for drugs for treating related diseases.

The present invention will be further illustrated below in combination with the drawings.

### BRIEF DESCRIPTION OF THE DRAWINGS

FIG. 1A shows genotyping of Axin1^{flox/flox} transgenic mice.

FIG. 1B shows genotyping of Agc1-CreER transgenic mice.

FIG. 2A shows micro-CT of the tibia of 6-month-old Axin1^{Agc1ER} mice comparing with Cre negative control mice.

FIG. 2B shows micro-CT of the cancellous bone of 6-month-old Axin1^{Agc1ER} mice comparing with Cre negative control mice.

FIG. 2C shows quantification of bone volume of 6-month-old Axin1^{Agc1ER} mice comparing with Cre negative control mice.

FIG. 2D shows quantification of trabecular number of 6-month-old Axin1^{Agc1ER} mice comparing with Cre negative control mice.

FIG. 2E shows quantification of trabecular thickness of 6-month-old Axin1^{Agc1ER} mice comparing with Cre negative control mice.

FIG. 2F shows quantification of trabecular separation of 6-month-old Axin1^{Agc1ER} mice comparing with Cre negative control mice.

FIG. 2G shows quantification of structure model index of 6-month-old Axin1^{Agc1ER} mice comparing with Cre negative control mice.

FIG. 2H shows histology of knee joint of 6-month-old Axin1^{Agc1ER} mice comparing with Cre negative control mice.

FIG. 2I shows histology of articular cartilage and growth plate of 6-month-old Axin1^{Agc1ER} mice comparing with Cre negative control mice.

FIG. 2J shows quantification of summed OASRI score of knee joint of 6-month-old Axin1^{Agc1ER} mice comparing with Cre negative control mice.

FIG. 2K shows quantification of average length of growth plate of 6-month-old Axin1^{Agc1ER} mice comparing with Cre negative control mice.

FIG. 3A shows Axin1 expression of knee joint of 6-month-old Axin1^{Agc1ER} mice comparing with Cre negative control mice.

FIG. 3B shows Axin1 expression of articular cartilage and growth plate of 6-month-old Axin1^{Agc1ER} mice comparing with Cre negative control mice.

FIG. 3C shows quantification of Axin1 expression of articular cartilage and growth plate of 6-month-old Axin1^{Agc1ER} mice comparing with Cre negative control mice.

FIG. 3D shows β-catenin expression of knee joint of 6-month-old Axin1^{Agc1ER} mice comparing with Cre negative control mice.

FIG. 3E shows β-catenin expression of articular cartilage and growth plate of 6-month-old Axin1^{Agc1ER} mice comparing with Cre negative control mice.

FIG. 3F shows quantification of β-catenin expression of articular cartilage and growth plate of 6-month-old Axin1^{Agc1ER} mice comparing with Cre negative control mice.

FIG. 3G shows Axin2 expression of knee joint of 6-month-old Axin1^{Agc1ER} mice comparing with Cre negative control mice.

FIG. 3H shows Axin2 expression of articular cartilage and growth plate of 6-month-old Axin1^{Agc1ER} mice comparing with Cre negative control mice.

FIG. 3I shows quantification of Axin2 expression of articular cartilage and growth plate of 6-month-old Axin1^{Agc1ER} mice comparing with Cre negative control mice.

FIG. 4A shows osteoclasts presentation of of growth plate of 6-month-old Axin1^{Agc1ER} mice comparing with Cre negative control mice.

FIG. 4B shows quantification of osteoclasts expression of growth plate of 6-month-old Axin1^{Agc1ER} mice comparing with Cre negative control mice.

FIG. 4C shows mRNA expression of OPG derived from Axin1 deficient BMS cells.

FIG. 5A shows Ki-67 expression of articular cartilage and growth plate of 6-month-old Axin1^{Agc1ER} mice comparing with Cre negative control mice.

FIG. 5B shows quantification of Ki-67 expression of articular cartilage and growth plate of 6-month-old Axin1^{Agc1ER} mice comparing with Cre negative control mice.

FIG. 5C shows Col-X expression of articular cartilage and growth plate of 6-month-old Axin1^{Agc1ER} mice comparing with Cre negative control mice.

FIG. 5D shows quantification of Col-X expression of articular cartilage and growth plate of 6-month-old Axin1^{Agc1ER} mice comparing with Cre negative control mice.

FIG. 5E shows MMP13 expression of articular cartilage and growth plate of 6-month-old Axin1^{Agc1ER} mice comparing with Cre negative control mice.

FIG. 5F shows quantification of MMP13 expression of articular cartilage and growth plate of 6-month-old Axin1^{Agc1ER} mice comparing with Cre negative control mice.

FIG. 5G shows TUNEL staining of articular cartilage and growth plate of 6-month-old Axin1^{Agc1ER} mice comparing with Cre negative control mice.

FIG. 5H shows quantification of TUNEL staining of articular cartilage and growth plate of 6-month-old Axin1^{Agc1ER} mice comparing with Cre negative control mice.

### DETAILED DESCRIPTION

Technical solutions in the present invention will be clearly and entirely described below in combination with the embodiments. Obviously, the described embodiments are only some embodiments of the present invention, but not all embodiments. Although representative embodiments of the present invention have been described in detail, ordinary technicians in the technical field to which the present invention belongs will understand that various modifications and changes can be performed for the present invention without departing from the scope of the present invention. Therefore, the scope of the present invention should not be limited to the implementation schemes, but should be restricted by the appended claims and equivalents thereof.

The experimental reagents used in the embodiments of the present invention are all commercially available conventional experimental materials.

### Embodiment 1

In traditional knee osteoarthritis animal models, mouse knee osteoarthritis model is relatively classic and widely used in knee osteoarthritis research. In this embodiment, mice were used as experimental subjects.

### I. Construction of Transgenic Animal Model

A method for constructing a transgenic animal model includes the following steps:

S1, Axin1^{flox/flox} transgenic mice are gifts from Professor Shu Bing (Shanghai University of Traditional Chinese Medicine), Agc1-CreER transgenic mice are purchased from Jackson Laboratories in the United States.

S2, Adult Axin1^{flox/flox} mice and Agc1-CreER mice are selected to mate.

When newborn mice grow to 2-3 weeks, 1-3 mm tail tips of the mice are taken for mice genotype identification; 51 μl lysis solution (Tris-Hcl (pH7.5) 2 μl, 0.5M EDTA (pH8.0) 20 μl, 5M NaCl 6 μl, 10% (M/V) SDS 20 μl, 10 mg/mlproteinase K 1 μl) is added into tissue needing lysis, incubating for 20 min in a water bath at 55° C. after vortex oscillation; then the sample is placed in a 95° C. or boiled water bath to be heated for 5 min, after the lysates product is thoroughly mixed evenly by vortex oscillation, supernatan is taken to obtain lysates product.

In the lysates product, PCR amplification is performed for Axin1 and CreER. The lysates product, H₂O, 2× Taq Plus Master Mix (Dye Plus), primer (forward), and primer (reverse) are configured in a ratio of 2.5:8:12.5:1:1 (μl), and PCR amplification is performed according to the program of 94° C. for 5 minutes, [94° C. for 30 seconds, 55° C. for 30 seconds, 72° C. for 30 seconds/kb], 72° C. for 7 minutes, and 4° C. A sequence of Axin1 forward primer is set forth in SEQ ID No. 1; a sequence of Axin1 reverse primer is set forth in SEQ ID No. 2; a sequence of CreER forward primer is set forth in SEQ ID No. 3; a sequence of CreER reverse primer is set forth in SEQ ID No. 4.

1.5 g agarose is dissolved in 100 ml TAE solution, and is heated in a 1000 W microwave oven for 3 minutes to prepare agarose gel; then add 10 μl nucleic acid dye is added into the solution, after the gel is solidified, the amplification product is added into the gel; then sizes of PCR product bands are detected by gel electrophoresis at 160V for 30 minutes; by the agarose gel electrophoresis, newborn mice corresponding to the lysates of Axin1 and CreER amplification bands are detected, as the Axin1^{Agc1ER} condition knockout mice.

S3, tamoxifen is injected into abdominal cavities of 2 months old Axin1^{Agc1ER} condition knockout mice, the injection amount is calculated based on the body weight of the Axin1^{Agc1ER} condition knockout mice, injecting 1 mg per 10 g, with the injection continuing for 5 days.

S4, biochemical indicators of the Axin1^{Agc1ER} condition knockout mice is regularly detected during modeling, and histological and histomorphometric analyses are performed.

S5, by inducing with tamoxifen, the Axin1^{Agc1ER} condition knockout mice experiences growth plate chondrocyte hypertrophy, ectopic ossification, and osteoarthritis diseases at 6 months old, thereby obtaining the transgenic animal model.

### II. Analysis for Transgenic Animal Model

The control group Cre consists of mice from the same litter that do not express Agc1ER.

Histological, micro-CT, and TRAP analyses are performed for the 6 months old Axin1^{Agc1ER} condition knockout mice and the control group Cre mice, the details are as follows.

Knee joints of the mice are fixed with 10% neutral buffered formalin (VWR, 16004-128). Then, by a μCT-35 cone beam scanner (Scanco Medical), a 55 kVp source, and current with a 145 resolution of 10 mA, and starting from a location where complete fusion of the tibial condyle is found, scanning is performed using 100 slices extending towards the proximal end. Reconstructed 3D images of each sample are evaluated under the same threshold.

The knee joints of mice are fixed, decalcified, and dehydrated using graded ethanol and xylene, and embed in paraffin. Starting from the inner side of the knee joints, midsagittal section μm spaces with a thickness of 3 μm are cut at three different horizontal planes (50°). Alcian blue/Orange G staining is performed on the knee joints of mice to perform morphological analysis.

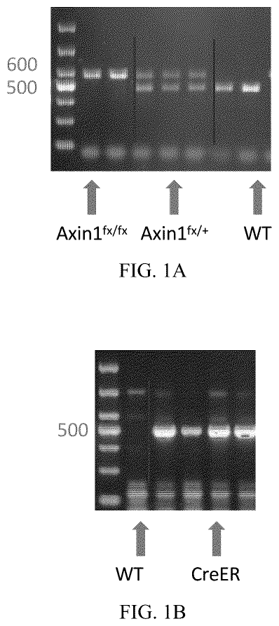

Genotype identification results of the Axin1^{Agc1ER} condition knockout mice are as shown in FIGS. 1A and 1B, wherein FIG. 1A shows Axin1 expression results of the Axin1^{Agc1ER} mice, the Axin1^{flox/flox} band is 600 bp, and the WT band is of a size about 500 bp; FIG. 1b shows Agc1ER expression results.

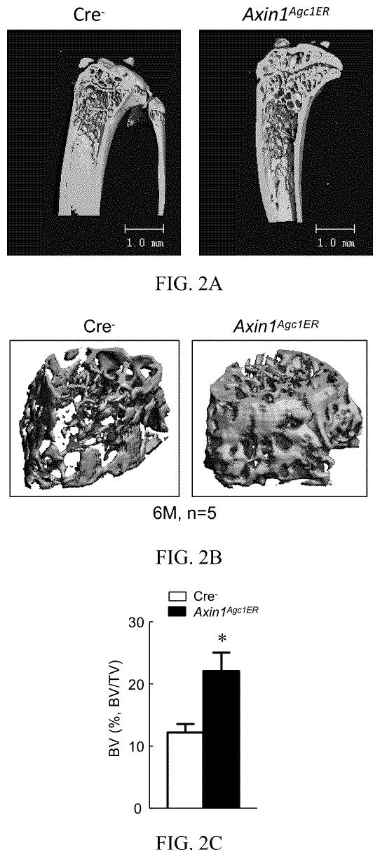

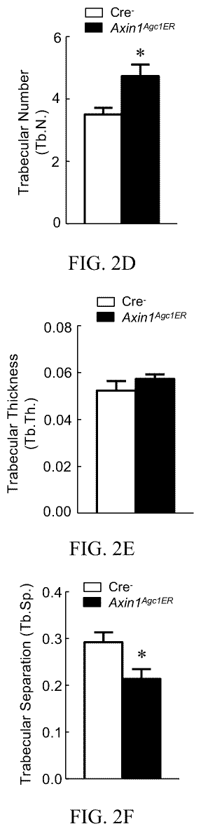

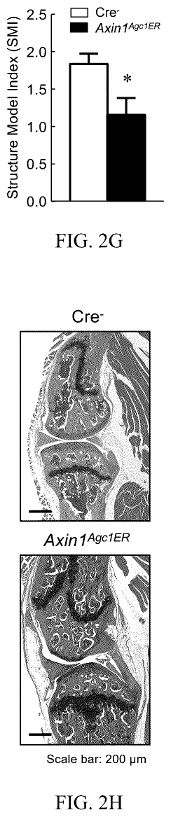

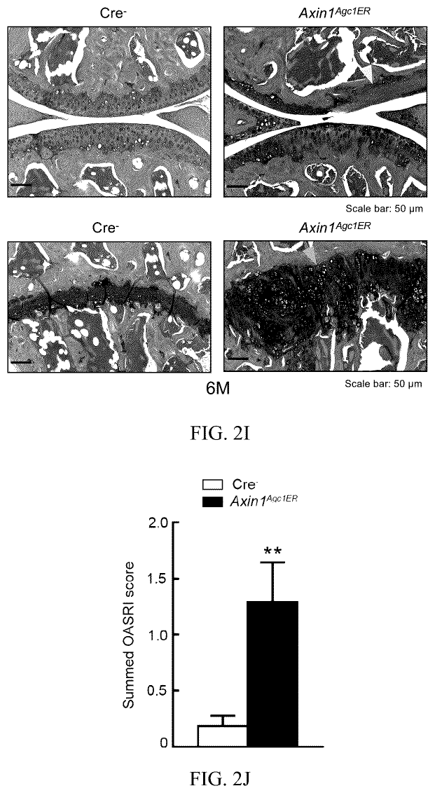

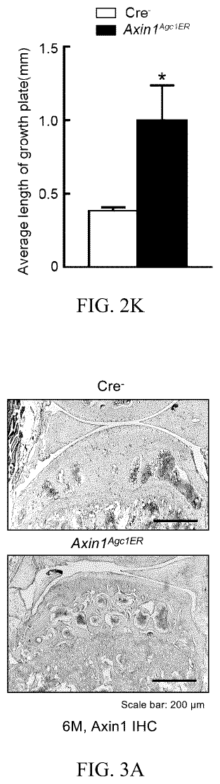

Histological and histomorphometric changes of the mice are as shown in FIGS. 2A-2K, wherein FIG. 2A and FIG. 2B show comparison results of bone mass changes of the mice, FIG. 2C and FIG. 2D show detection results of bone volume (BV) and trabecular quantity (Tb. N.) of the mice, FIG. 2E show detection results of trabecular thickness (Tb. Th.), and FIG. 2F and FIG. 2G show trabecular separation (Tb. Sp.) and structural model index (SMI) of the mice. Compared with the control group, in the 6 months old Axin1^{Agc1ER} condition knockout mice, both the bone volume and the number of trabeculae (Tb. N.) increase, there is no significant difference in the trabecular thickness (Tb. Th.). and both the trabecular separation (Tb. Sp.) and the structural model index (SMI) decrease. FIG. 2H and FIG. 2I show Alcian blue/orange G staining, showing slight articular cartilage degeneration in growth plates of femurs and tibias of the 6 months old Axin1^{Agc1ER} condition knockout mice. The medial femoral condyle is analyzed at three levels of the joint and the total OARSI score for the entire joint is provided, the results are shown in FIG. 2J, the total OARSI score of the 6 months old Axin1^{Agc1ER} condition knockout mice is significantly higher than that of the control group. FIG. 2K shows the average tibial growth plate width of the mice, the average tibial growth plate width of 6 the 6 months old Axin1^{Agc1ER} condition knockout mice is significantly wider than that of the control group.

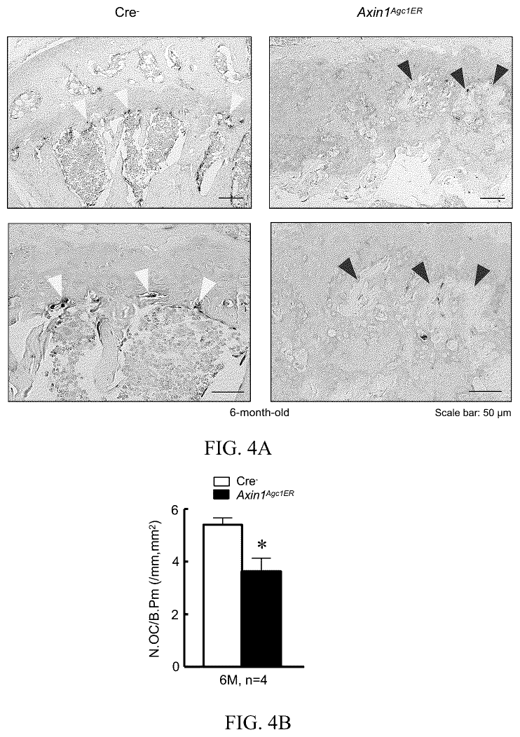

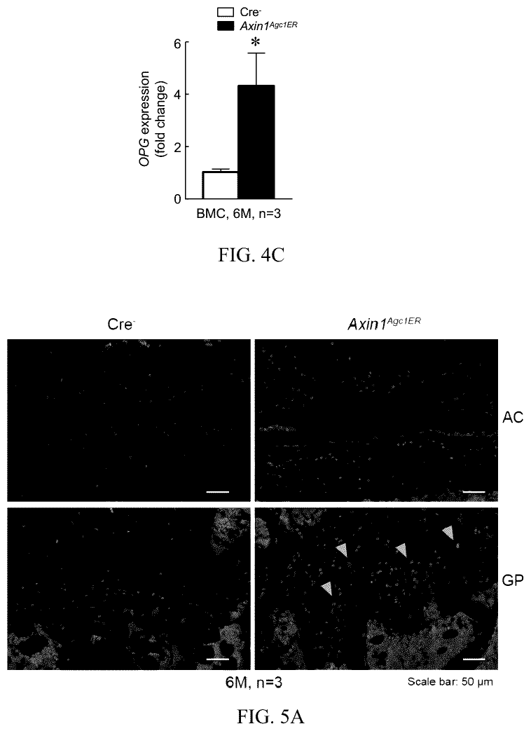

TRAP staining is performed on a midsagittal plane slider with a thickness of 3 μm, n≥4. First, a glass slide is baked overnight at 60° C., then dewaxed and re-hydratized. Next, the glass slide is incubated in dark at 37° C. for 1 hour with TRAP staining buffer liquid (sodium acetate, sodium tartrate, solid red purple LB salt, naphthol AS-MX, dimethylformamide, MnCl2). Then the slices are re-stained with CAT hematoxylin (Biocare Medical, CATHE-GL) and covered with glycerol gelatin (Sigma, GG1-15 ML). The Trap detection results are as shown in FIG. 4A. OsteoMeasure software (OsteoMetrics, Inc., Atlanta, GA, USA) is used to measure osteoclasts below the growth plate, and bone circumference (N.Oc/B.Pm) is used as a reference for expression, the results are as shown in FIG. 4B and FIG. 4C. The 6 months old Axin1^{Agc1ER} condition knockout mice shows reduced osteoclast formation and appearance of ectopic bones, and OPG expression significantly increases in Axin deficient BMS cells.

Immunohistochemical (IHC) and immunofluorescence (IF) analyses are performed for the 6 months old Axin1^{Agc1ER} condition knockout mice and the control group Cre mice, the details are as follows.

The detected IHC and IF indicators of the mouse model provided by the present invention includes expression of Ainx1, Aixn2, β-catenin, Col-X, MMP13, and Ki-67 of the 6 months old Axin1^{Agc1ER} condition KO mice. IHC and IF are also performed on a midsagittal plane slider with a thickness of 3 μm, n≥3. First, a glass slide is baked overnight at 60° C., then dewaxed and re-hydratized. Then the glass slide is incubated with Antigen unmasking solution (95° C., 5-10 minutes), 30% hydrogen peroxide (10 minutes), 0.5% Triton 100 (15 minutes), primary antibody (12 hours) and secondary antibody (1 hour), coloration by DAB (10-300 seconds), re-stained by hematoxylin and ammonia, soaking in alcohol and xylene, and sealing with neutral resin.

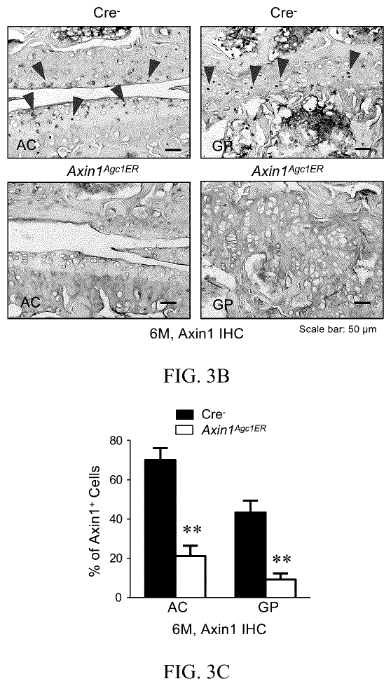

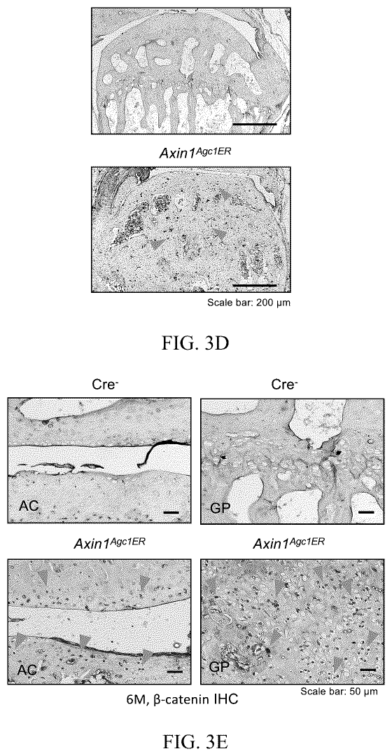

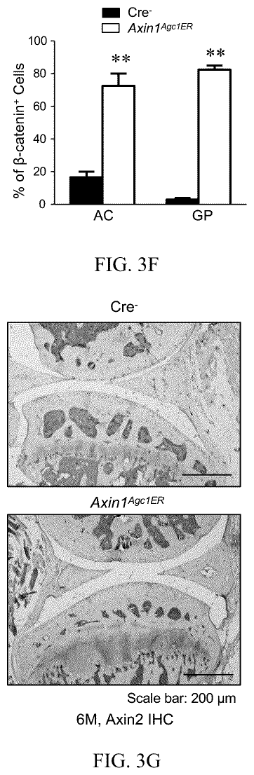

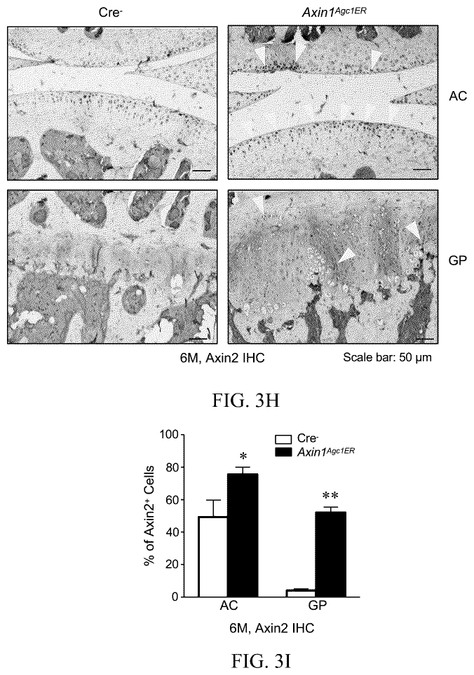

FIGS. 3A-3I shows immunohistochemical (IHC) analysis results of Axin1, β-catenin, and Axin2 protein expression, *P<0.05, ** P<0.01, n=3 mice/group. From FIGS. 3A-3C, it can be seen that in acticular cartilage and growth plate cartilage (Axin1+ cells) of the 6 months old Axin1^{Agc1ER} condition knockout mice, the levels of Axin1 proteins are all eliminated. From FIGS. 3D-3F, it can be known that the β-catenin levels of acticular cartilage and growth plate cartilage (β-catenin+ cells) of the 6 months old Axin1^{Agc1ER} condition knockout mice are all elevated. In FIGS. 3G-3I, the Axin2 protein levels of acticular cartilage and growth plate cartilage (Axin2+ cells) of the 6 months old Axin1^{Agc1ER} condition knockout mice are elevated.

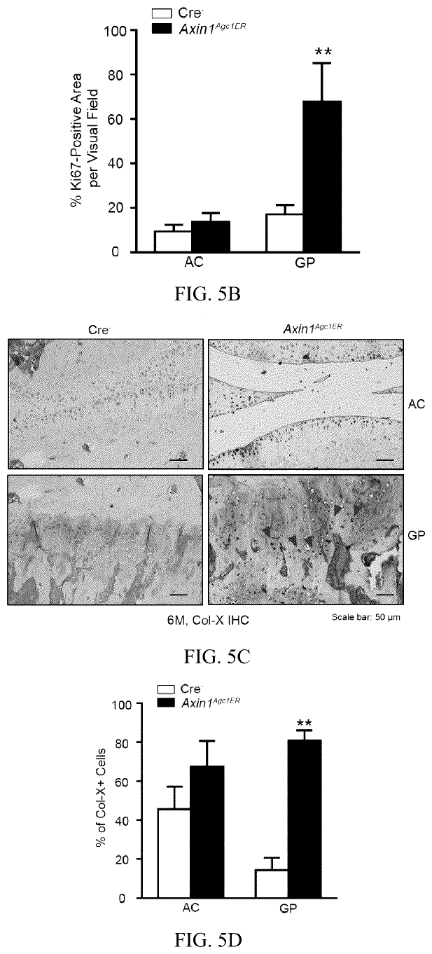

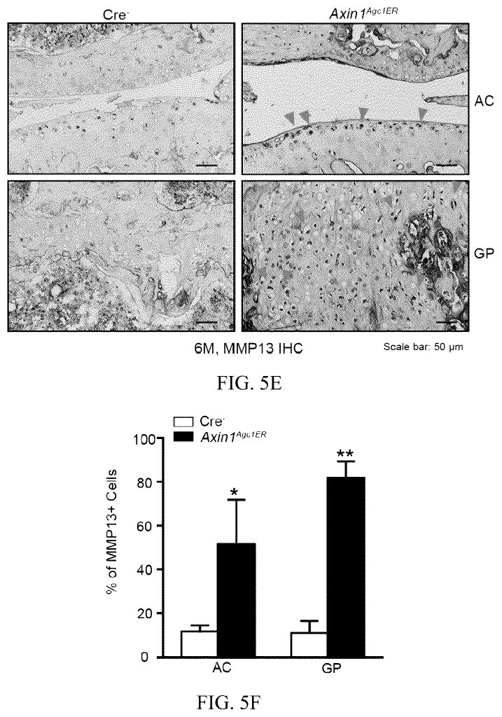

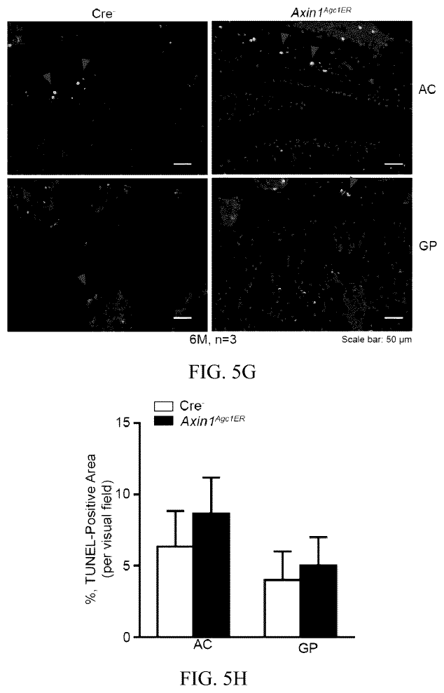

Changes of proliferation, differentiation, and apoptosis of cartilage cells of the 6-month-old Axin1^{Agc1ER} condition knockout mice are as shown in FIGS. 5A-5H (*P<0.05, ** P<0.01, n=3 mice/group). Ki67 positive cells in the expanded growth plate of the 6-month-old Axin1^{Agc1ER} condition knockout mice increase significantly (FIG. 5A-FIG. 5B); at growth plate cartilage, ColX protein expression increases (FIG. 5C-FIG. 5D); at articular cartilage and growth plate cartilage, MMP-13 protein expression increases (FIG. 5E-FIG. 5F); in the expanded growth plate, TUNEL staining positive apoptotic cells have no significant changes (FIG. 5G-FIG. 5H).

What is claimed is:

1. A method for constructing a transgenic animal model, comprising the following steps:
S1, constructing Axin1^{flox/flox} transgenic mice and Agc1-CreER transgenic mice;
S2, selecting adult Axin1^{flox/flox} mice and Agc1-CreER mice to mate, and producing Axin1^{Agc1ER} condition knockout mice by mating;
S3, injecting tamoxifen into abdominal cavities of adult Axin1^{Agc1ER} condition knockout mice, with the injection continuing for 5 days;
S4, regularly detecting biochemical indicators of the Axin1^{Agc1ER} condition knockout mice during modeling, and performing histological and histomorphometric analyses;
S5, when an Axin1^{Agc1ER} condition knockout body simultaneously experiences growth plate chondrocyte hypertrophy, ectopic ossification, and knee joint cartilage degeneration syndrome, obtaining the transgenic animal model.

2. The method for constructing a transgenic animal model according to claim 1, wherein the step S2 specifically comprises: mating adult female Axin1^{flox/flox} transgenic mice with adult male Agc1-CreER transgenic mice, or mating adult male Axin1^{flox/flox} transgenic mice with adult female Agc1-CreER transgenic mice; when newborn mice grow to 2-3 weeks, taking 1-3 mm tail tips of the mice for mice genotype identification; adding lysis solution into tissue needing lysis, and incubating in a water bath after vortex oscillation; after thoroughly mixing evenly by vortex oscillation, taking supernatan to obtain lysates product; performing PCR amplification for Axin1 and CreER in the lysates; by agarose gel electrophoresis, detecting newborn mice corresponding to the lysates of Axin1 and CreER amplification bands as the Axin1^{Agc1ER} condition knockout mice.

3. The method for constructing a transgenic animal model according to claim 2, wherein the lysis solution comprises 2 μl Tris-Hcl with a pH of 7.5, 20 μl 0.5M EDTA with a pH of 8.0, 6 μl 5M NaCl, 20 μl SDS with a mass to volume ratio of 10%, and 1 μl proteinase K with a concentration of 10 mg/ml.

4. The method for constructing a transgenic animal model according to claim 2, wherein primer used in the PCR amplification include Axin1 forward primer, Axin1 reverse primer, CreER forward primer, and CreER reverse primer; a sequence of the Axin1 forward primer is set forth in SEQ ID No. 1; a sequence of Axin1 reverse primer is set forth in SEQ ID No. 2; a sequence of CreER forward primer is set forth in SEQ ID No. 3; a sequence of CreER reverse primer is set forth in SEQ ID No. 4.

5. The method for constructing a transgenic animal model according to claim 1, wherein in the step S3, the injection amount of tamoxifen is calculated based on the body weight of the Axin1^{Agc1ER} condition knockout mice, injecting 1 mg per 10 g.

6. The method for constructing a transgenic animal model according to claim 1, wherein in the step S4, the biochemical indicators comprise expression levels of Ainx1, Aixn2, β-catenin, COl-X, MMP13, and Ki-67 in the bodies of the Axin1^{Agc1ER} condition knockout mice.

7. The method for constructing a transgenic animal model according to claim 1, wherein in the step S4, the histomorphometric analyses comprise expression of micro-CT and TRAP staining of the Axin1^{Agc1ER} condition knockout mice.

8. Application of a transgenic animal model obtained by the method according to claim 1 in screening drugs for the treatment of growth plate chondrocyte hypertrophy, ectopic ossification, and osteoarthritis diseases.

9. Application of a transgenic animal model obtained by the method according to claim 1 in systematic research on the pathogenesis of growth plate chondrocyte hypertrophy, ectopic ossification, and osteoarthritis.


SEQUENCE LISTING (4 sequences)

SEQ ID NO: 1 - DNA, 21 bp, synthetic construct
  source 1..21: mol_type = other DNA
tgaactcttgatcaggtcttg

SEQ ID NO: 2 - DNA, 22 bp, synthetic construct
  source 1..22: mol_type = other DNA
tatgatctttggtccctttctg

SEQ ID NO: 3 - DNA, 18 bp, synthetic construct
  source 1..18: mol_type = other DNA
ctggttggacagtgcccg

SEQ ID NO: 4 - DNA, 22 bp, synthetic construct
  source 1..22: mol_type = other DNA
tgccaggtttggtcagtaagcc